(19) United States
(12) Patent Application Publication
(10) Pub. No.: US 2016/0324462 A1
(43) Pub. Date: Nov. 10, 2016

(54) METHOD AND SYSTEM FOR PROVIDING FEEDBACK AUTOMATICALLY ON PHYSIOLOGICAL MEASUREMENTS TO A USER

(71) Applicant: FIRSTBEAT TECHNOLOGIES OY, Jyväskylä (FI)

(72) Inventors: Kaisa HÄMÄLÄINEN, Jyväskylä (FI); Joni KETTUNEN, Jyväskylä (FI); Satu TUOMINEN, Jyväskylä (FI); Ilkka YLIKULJU, Jyväskylä (FI)

(73) Assignee: Firstbeat Technologies Oy, Jyväskylä (FI)

(21) Appl. No.: 15/107,692

(22) PCT Filed: Feb. 2, 2015
(86) PCT No.: PCT/FI2015/050067

§ 371 Date: Jun. 23, 2016

(30) Foreign Application Priority Data
Jan. 31, 2014 (FI) 20145105

(51) Int. Cl. A61B 5/00; G06F 19/00; A61B 5/0205; A61B 5/01; A61B 5/11

(52) U.S. Cl. CPC A61B 5/486; A61B 5/742; A61B 5/7264; A61B 5/01; A61B 5/1112; A61B 5/02055; A61B 5/0022; G06F 19/3406; A61B 2562/0219; A61B 2560/0252; A61B 5/02416

(57) ABSTRACT

A method and system for providing computer-aided feedback to a person with the aid of a monitor and expert system. The providing of feedback includes monitoring a state, such as heart rate, of the person continuously by a monitor for at least a portion of a day, determining at least one temporal context from the analysed measurement data, classifying the value of each preselected variable into one said value range and picking from each feedback series a feedback corresponding to this value range, arranging the picked feedbacks into an order according to the priority rules, thus forming a group of consecutive feedbacks, and displaying the consecutive feedbacks, picked from the group of consecutive feedbacks, for viewing by the person.

The invention relates to a method for providing computer-aided feedback to a person, with the aid of monitoring and an expert system, in which method the providing of feedback takes places in the following steps:
the state of the person is monitored continuously, with the monitoring being recorded as measurement data and the said monitoring including the measurement of heart rate using a first sensor, and an optional amount of other input,
analysing the recorded measurement data, and displaying the feedback to the person using a chosen device.

The invention also relates to a corresponding system.

To prevent various illnesses it is important for sufficient measurement data to be available on the physiology of a person. Simply measuring and showing the results in a numerical form often does not serve the consumer. When physiology is measured, it is important that the results are interpreted correctly and concrete feedback on this is obtained. The interpretation of physiological data is challenging and, in a normal situation, an expert's know-how is required in the interpretation.

Various feedback systems, in which the user is provided with feedback, are known from the prior art. These feedback systems often demand a great deal of manually entered data, and do not use physiological data. The feedback systems according to the prior art often provide the user with information or feedback that is unarticulated and difficult to understand, and thus do not serve the user in the best possible way.

Patent application US 2013/0316313 A1 discloses a method, which provides an internet-based lifestyle management service, which assists in detecting flaws in lifestyle and in guiding towards goals set by the user. The system includes an interactive calendar, in which the user makes diary entries concerning, among other things, exercise, sleep time, work time, and meals. The system is based on data entered manually by the user and not, for example, on physiological data. The goals chosen by the user relate to the duration of various daily tasks and the feedback on this is highly simplified, being based on the duration of the various tasks. The method disclosed in patent application US 2013/0143182 A1 is also based on a similar manual entry of activities, and gives points, which show the possibility of achieving goals, as feedback to the user after each manually entered datum. In this situation, the feedback to the user is also highly simplified. Such methods do not, however, give sufficiently accurate and objective information on the user's state of health.

Various feedback systems using physiological measurement are also disclosed in publications US 2010/0227302 A1, US 2013/0142182 A1, and U.S. Pat. No. 8,275,635 B2. However, all of these have the drawback that they do not provide clear and simplified feedback on the basis of physiological measurement and require manual work by the user in order to provide feedback. In publication US2007/0299330 (Couronne), an acceleration sensor is used to improve the reliability of measurement by a PPG sensor.

Known from the prior art is the applicant's application WO 2013/068650 A2, which discloses a method for determining the physiological state of a person; US2014/0288448 A1, which is “herein incorporated by reference” (US). In this method, physiological measurements are performed on the person, on the basis of which vectors are defined for selected variables to be measured. The vectors are situated in a table containing various feedback series, from which with the aid of correlation equations a correlation value is calculated for how well the vector of a variable correlates with a discrete physiological state. Finally, the correlation values are summed and, on the basis of comparison, the person's physiological state and the related feedback are obtained as a result. However, such a method gives as a final result only fixed feedback relating to a specific state. The method is not suitable for providing feedback on several matters in greater detail, because there is no actual feedback system in it.

The invention is intended to create a more highly-developed method than the methods of the prior art for providing feedback to a person automatically, in such a way that the amount of feedback to be provided is limited to comprise only the essential feedbacks. A great challenge to automatic feedback is that it is difficult to avoid giving erroneous feedback from only processing primary information obtained from even errorless heart-rate information. The characteristic features of the present invention are stated in the accompanying claim 1. The invention is also intended to create a more highly-developed system than systems of the prior art for providing feedback automatically to a person, in such a way that the amount of feedback to be provided is limited to comprise only the essential feedbacks. The characteristic features of this invention are stated in claim 12.

Erroneous interpretations can be effectively prevented with the aid of a second measurement input. Preferably both motion detection and positioning (GPS) are also utilized to accurately differentiate the context. Both are preferably used both to improve the reliability of the heart-rate measurement and to differentiate the contexts. The accurate differentiation of contexts avoids a considerable number of erroneous interpretations and the erroneous feedbacks deriving from them. In continuous monitoring, the significance of positioning in differentiating contexts increases, as the system recognizes the regular locations of the person (home, workplace, gym, etc.) increasingly well.

The method and system according to the invention are intended to automatically provide concrete, verbal feedback from physiological data directly to the consumer, either in real time or through later analysis. Because it is often possible to give a great many different kinds of feedback from the same measurement, it is important to prioritize the feedbacks so that they are appropriate, as it is not sensible to give, for example, fifty different feedbacks from a single day. This method and system therefore include the smart prioritization of feedbacks, in addition to detecting various kinds of situation from physiological data.

The intention of the method according to the invention can be achieved by means of a method for providing computer-aided feedback to a person with the aid of monitoring and an expert system, which expert system includes feedback series that are used by means of software and relate to a temporal context and a set of rules implemented by means of software for selecting the feedback series of the feedback. The set of rules includes priority rules for prioritizing the selected feedbacks consecutively and limit rules for selecting the number of feedbacks, to be shown at one time to the person, from the consecutive feedbacks. In the method, each feedback series comprises a group of mutually exclusive preselected feedbacks, each feedback relating to the unique value range of a preselected variable. In the method, the provision of feedback takes place in the following steps, in which the state of the person is monitored continuously, recording the monitoring as measurement data, the said monitoring including the measurement of heart rate using a first sensor and an optional amount of other input. The recorded measurement data is analysed in order to determine the values of preselected variables, at least one temporal context is defined from the analysed measurement data, and the feedback series to be used are chosen according to the defined contexts. The value of each preselected variable is classified in a corresponding value range, a feedback corresponding to this value range from each feedback series is picked, and the picked feedbacks are arranged in sequence according to the priority rules, forming a group of consecutive feedbacks. A number of picked consecutive feedbacks according to the limit rules is shown to the person, using a chosen device. Using such a method, concise and condensed feedback on their physiological state can be produced automatically for a person from a large number of preselected feedback alternatives.

The variable relating to a feedback series is preferably a vector, comprising two or more scalar variables. This means that the variable can consist of several variables, so that the vector has several dimensions consisting of several individual scalar variables. Often the amount of measurement error forms an extra condition for the use of any variable whatever. The combination [measurement error, variable] can be considered a vector. Often there is also acceleration/positioning data to eliminate measurement errors, and also to accurately separate contexts. The reliability of individual feedbacks improves when the circumstances of the heart-rate data are defined.

The variable can be continuous or discrete, i.e. when it is continuous it can receive, for example, the selected value ranges 0-15%, 16-50%, 51-100%, or when discrete it can receive, for example, the values 1 or 0. The value range contracts to become point-like.

The limit rules can include logic dependent on the selected feedback device. In other words, for example when using a device with a smaller display, a numerically smaller amount of feedback is shown to the person than when, for example, using the screen of a tablet.

In the method, feedback directions can be formed to be selected by the user, each of which feedback directions includes feedback series preselected according to it. By selecting a feedback direction, the priority rules can be altered in such a way that feedbacks relating more closely to a specific feedback direction are given a higher priority than the feedbacks of the other feedback directions.

The feedbacks selected in each feedback direction are preferably set feedback-direction-specifically in the priority sequence. Thus, the most important feedbacks in terms of the feedback direction are displayed first.

Each feedback is picked on the basis of a preselected rule. Error detections are reduced through the joint effect of two or more variables.

The measurement data can be analysed in the following steps in order to find physiological states, in which the measurement data is corrected in order to eliminate disturbances and is pre-processed, the value of the variable is created on the basis of the corrected and pre-processed measurement data, stationary states are segmented from the measurement data, states of physical activity are identified, the measurement data is compared to known other physiological states, and the physiological states are identified. After the identification of states of physical activity, variations in the activity level due to other causes than exercise, such as moments of mental load or recovery, can be separated. Thus physiological states can be reliably defined using preselected criteria.

The other input preferably includes positioning and/or acceleration measurement of the person. The temporal context can then be identified on the basis of positioning or acceleration, for example, the temporal context “swimming”, if the person is positioned in a swimming pool. One feedback series preferably comprises a conditional-statement group, in which only one conditional statement can be realized at one time. The feedbacks in the conditional-statement group are thus mutually exclusive.

The conditional-statement groups can be set to be realized consecutively, so that consecutive feedbacks will be obtained in this order. In other words, the consecutive placing of the conditional-statement groups can, at the same time, form the priority order of the feedbacks.

According to one embodiment, the heart rate is measured using an ECG sensor. Measurement data obtained with the aid of an ECG sensor is accurate and contains very few errors.

The method according to the invention can be implemented using a computer program in a personal computer, to a heart-rate meter (wrist-worn computer), ECG, or PPG device. Generally the implementation consists of a processing unit, a terminal device, software, and at least one device for inputting data. A particularly good totality can be obtained with the aid of a PPG wrist-worn device, a smart phone equipped with a large display, and a positioning device. The PPG wrist-worn device and the smart phone are connected to each other by a wireless local connection (such as Bluetooth). These can be easily carried by the person being examined. The smart phone is not needed continuously, because there is usually a buffer memory in the PPG device (as usually in other heart-rate meters too). AirDrop and WiFi links can also be used. The positioning device (e.g. GPS) has a double importance. It can be used to improve the reliability of the variables and to define the context accurately.

The physiological measurement is preferably performed continuously from the person's daily life, i.e. outside laboratory conditions, in other words from random conditions depicting the person's activities. Here the term continuous measurement should be understood from a physiological viewpoint. Technically, the measurement can be discontinuous. A continuous depiction of the physiological state is obtained as the end result. Depending on the case, i.e. the variable being examined, measurement once in 30 minutes can be enough, if it is determined, for example, whether or not it is a workday. On the other hand, if internal parameters of the heart rate are being investigated, the measurement frequency must be high at least periodically.

Software can be used to automatically detect the apparatus and user interface for showing feedback. The detection data can be used together with limit rules to limit the feedback according to the device used to display the feedback.

The intention of the system according to the invention can be achieved by means of a computer-aided system based on monitoring for providing feedback to a person, which system includes a monitor for continuously monitoring the state of the person and recording the data obtained, and an apparatus comprising processing means for processing by software feedback series relating to a temporal context according to a preset set of rules in order to select the feedbacks. In addition, the system includes a user interface for input by the user and for showing the selected feedbacks to the person. The set of rules includes priority rules for prioritizing the selected feedbacks consecutively and limit rules for selecting from the consecutive feedbacks the number of feedbacks to be shown to the person at one time, in which each feedback series comprises a group of mutually exclusive feedbacks, each feedback relating to the unique value range of a preselected variable. The processing means are arranged to analyse the recorded measurement data in order to determine the values of the preselected variables, to define at least one temporal context from the analysed measurement data, and to select the feedback series to be used, according to the defined contexts. In addition, the processing means are arranged to classify the value of each preselected variable in a value range corresponding to it, to pick feedback corresponding to this value range from each feedback series, to arrange the picked feedbacks in an order according to the priority rules thus forming a group of consecutive feedbacks, and to show according to the limit rules a number of picked consecutive feedbacks to the person, with the aid of the user interface. Such a system permits prioritized feedback to be given to the person automatically, without separate analysis by a physiological expert.

The processing means preferably include software means for the person to select the feedback direction and for selecting preselected feedback series according to the selected feedback direction. Thus the user need only select the emphasis with which they wish to receive feedback from a measurement period.

The monitoring device for monitoring a person can include positioning means. With the aid of the positioning means, specific locations can be tied to specific contexts, such as, for example, a yoga studio to the context “yoga training”.

The monitoring device for monitoring a person preferably includes an ECG sensor for measuring heart rate. With the aid of the ECG sensor reliable measurement data is produced concerning the person's heart rate.

The system preferably includes a data-transfer network and a server for maintaining a database in the internet. Thus the processing means can also be separate from the monitor, for example, as a cloud service in the internet.

The use of the method and system according to the invention permits feedback to be automatically provided to a person without using a physiological expert to interpret the measurement data. In addition, the method and system according to the invention permit feedback to be limited to essential matters, in the case of the provision of feedback, according to an emphasis chosen by the person themselves, taking into account the limitations imposed by the apparatus used for providing feedback. Though the steps of the method in the Claims and examples are presented in a specific order, it should be understood that the steps of the method can also be applied in a different order.

In the following, the invention is described in detail with reference to the accompanying drawings depicting some embodiments of the invention, in which

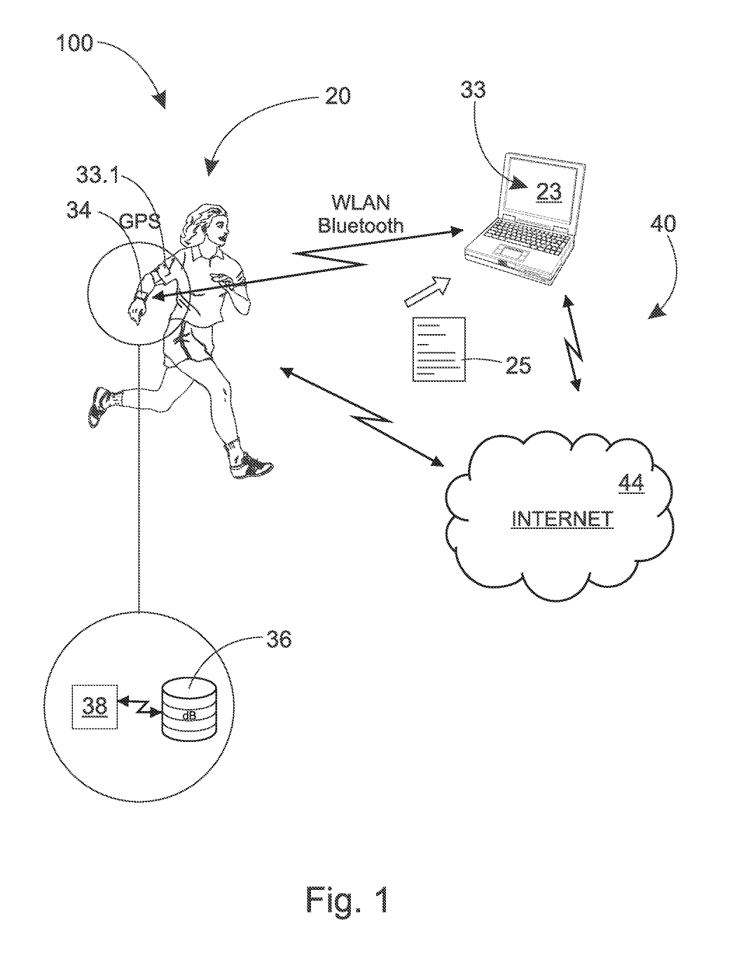

FIG. 1 shows a schematic view of the devices of the system according to the invention,

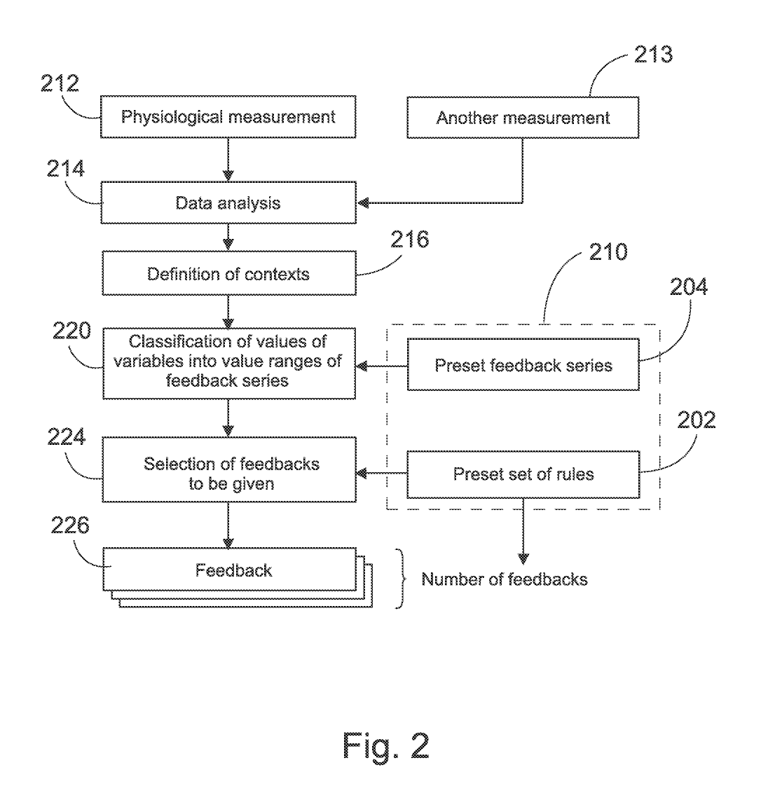

FIG. 2 shows the method according to the invention as a simplified flow diagram,

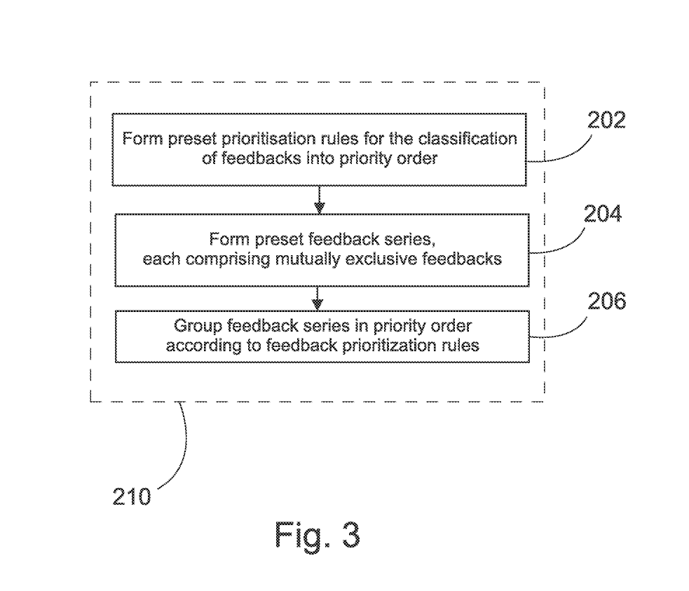

FIG. 3 shows a flow diagram of the steps of setting the basic data of the method according to the invention,

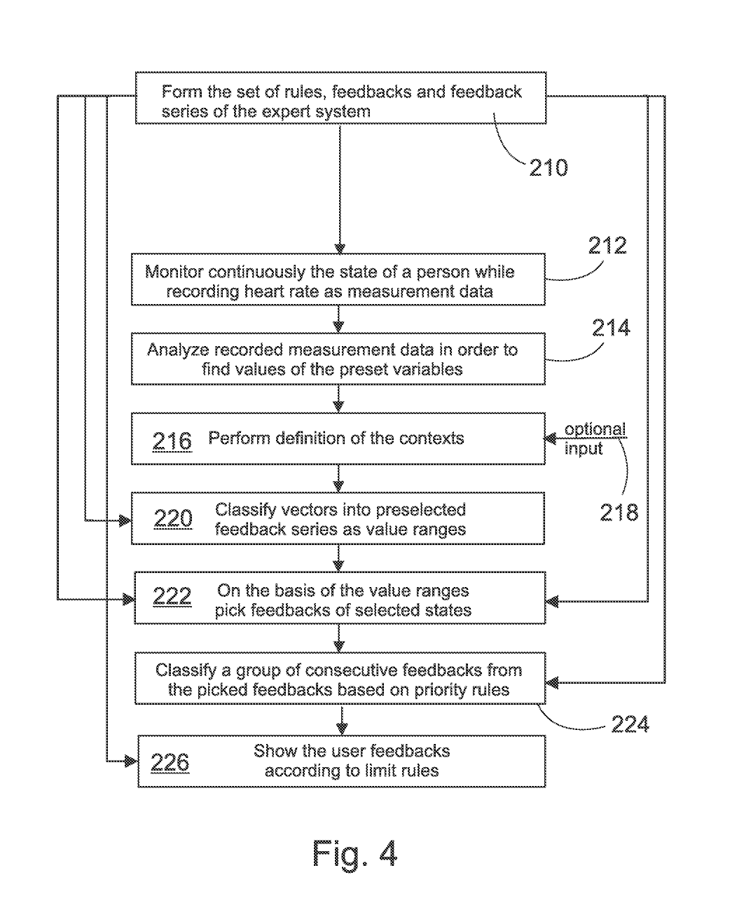

FIG. 4 shows a flow diagram of the step of the method according to the invention, after setting the basic data,

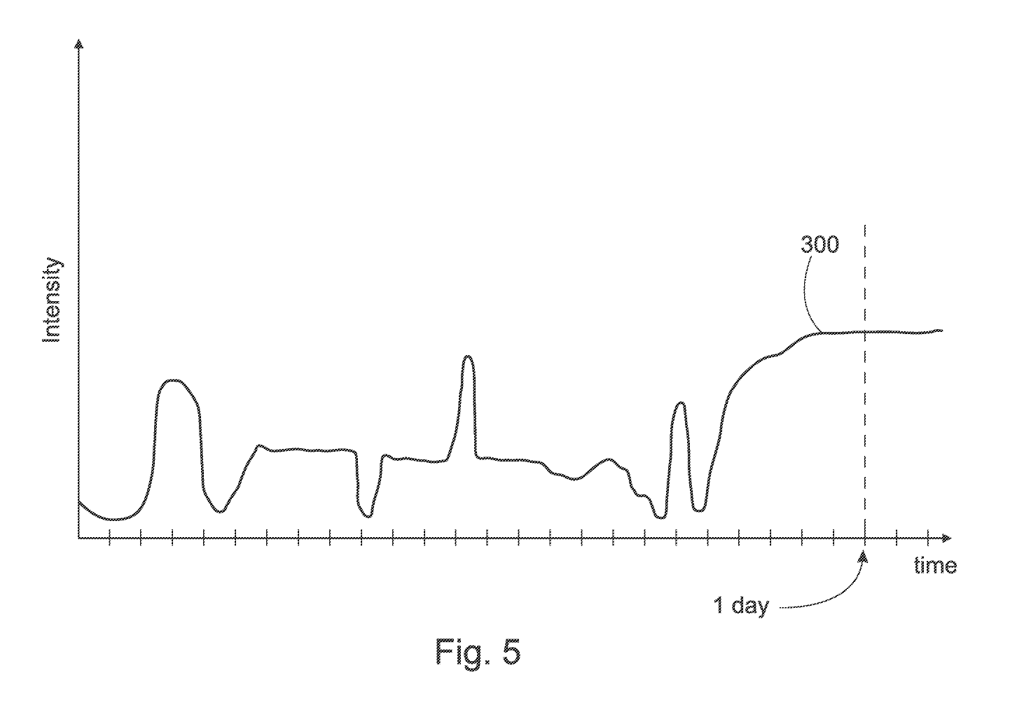

FIG. 5 shows a heart-rate curve measured using the method according to the invention,

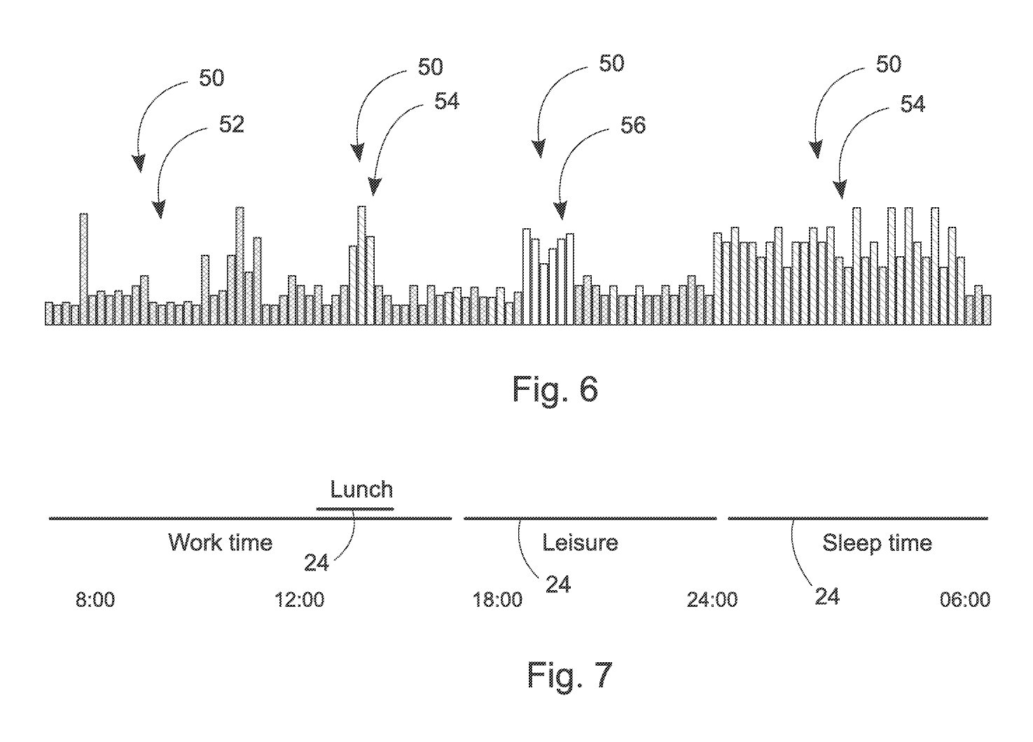

FIG. 6 shows the physiological states formed from the heart-rate curve measured using the method according to the invention,

FIG. 7 the contexts defined from the heart-rate curve measured using the method according to the invention,

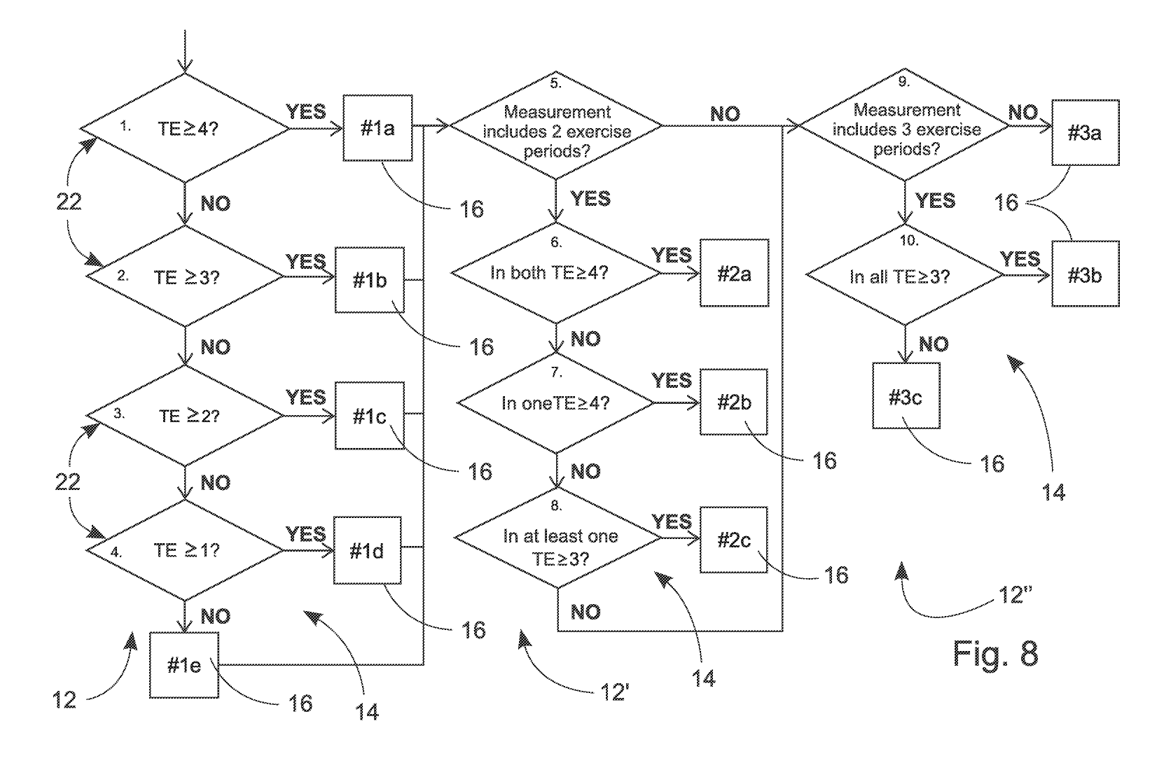

FIG. 8 shows the exercise-weighted feedback series according to one embodiment of the method according to the invention,

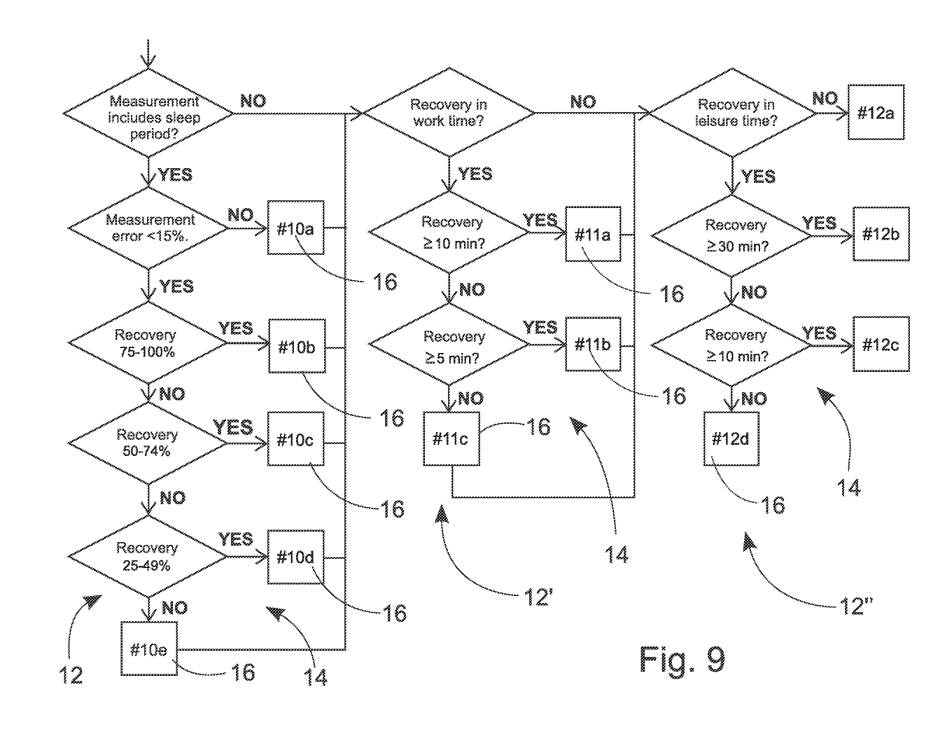

FIG. 9 shows the stress-weighted feedback series according to one embodiment of the method according to the invention,

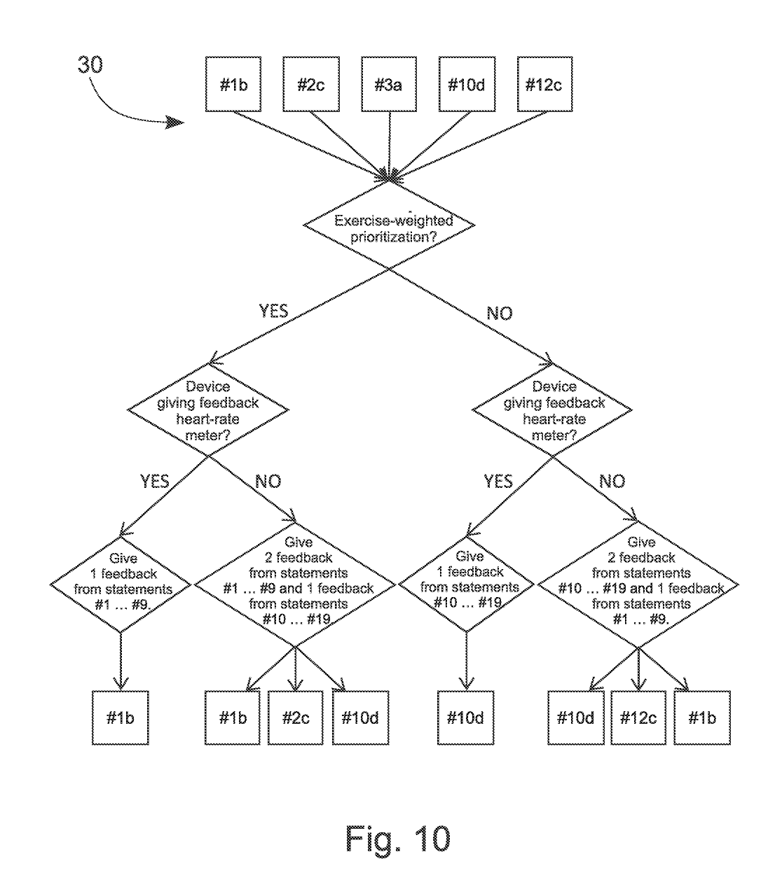

FIG. 10 shows the selection of the feedback to be shown according to the invention from a prioritized group,

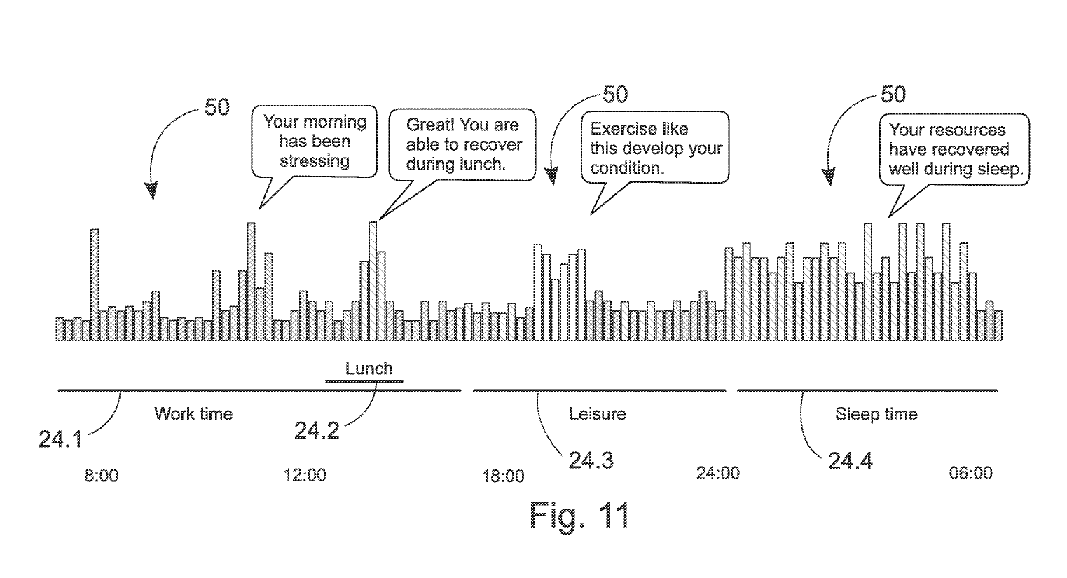

FIG. 11 shows the feedback to be shown to the person.

In the present application, the following terms are used:

| | |
|---|---|
| Feedback series | a group containing one or more questions, in which the question relates to |
| | at least one variable or state data to be monitored in the |
| | physiological analysis. |
| Value range | the possible range of variation of the value of a variable. The complete |
| | variation range is divided into the selected number of value |
| | ranges, each forming a unique class. |
| Context | a temporal period, which depicts a specific activity of the person during a |
| | measurement period. |
| Priority rules | rules, on the basis of which the feedbacks are placed in a consecutive order of |
| | importance. |
| Limit rules | rules that define the number of feedbacks selected to be shown to the |
| | person. For example, five feedbacks can be shown on the user |
| | interface of a tablet, whereas two are shown on the user interface |
| | of a heart-rate meter. |
| Feedback | a short verbal or visual description, which is based on |
| | measurement data and which summarizes, for example, the |
| | success of the person in achieving a goal, or which defines the |
| | measures needed to achieve a goal. |
| Continuous measurement | a variable, such as the completely continuous or partly periodic |
| | monitoring of the heart rate with the aid of a chosen monitoring |
| | device, which produces a continuous depiction of the |
| | physiological state and/or conditions of the person being |
| | examined. |
| Measurement period | a temporal period, during which the measurement of heart rate |
| | and other variables is performed with the aid of a chosen |
| | monitoring device. |
| Heart-rate measurement | the collection of heart-rate data directly or indirectly, for example, |
| | with the aid of a heart-rate meter, ECG-measurement, optical |
| | measurement (PPG), or similar. |

FIG. 1 shows a simplified form of implementation of the computer-aided system 100 according to the invention. In the system 100, the state of a person 20 is monitored with the aid of a monitor 34, preferably as a continuous measurement, and the monitor 34 records the data obtained. The monitor can preferably be a heart-rate meter, in which an accessory can be used to measure an optional variable, such as acceleration, position, or the temperature of the person or the environment.

The measurement can be in various ways periodic, for example, one minute of measurement and a five minutes break, or in the ratio 5 min/60 min. So much data is always obtained for the desired purpose that the physiological and state depictions are continuous in the range of the examined variable.

The optional variable can be, for example, acceleration or positioning data. In addition to the monitor 34, the system 100 includes an apparatus comprising processing means 38 and a database 36 for the processing by software of feedback series 12, 12′, 12″ relating to a temporal context 24, according to a preset set of rules, in order to select feedbacks 18. Further, the apparatus includes a user interface 23 for input by the user and for displaying selected feedbacks to the person 20. The set of rules includes priority rules for prioritizing the selected feedbacks consecutively, and limit rules for selecting the number of feedbacks 18 to be shown to the person 20 at one time from the consecutive feedbacks 18, in which each feedback series 12, 12′, 12″ comprises a group of mutually exclusive preselected feedbacks 18, each feedback 18 relating to the unique value range of a preselected variable. The term database 36 should be understood widely. It refers to the recording of feedback series and sets of rules in some appropriate manner, also directly in program code.

The processing means 38 can be situated in the monitor 34 as in FIG. 1, but they can also be in a separate device, for example, in a computer, in a mobile phone, in a tablet, or as a service in the internet 44. The user interface 23 for showing feedback is preferably in the same device, by which physiological data is measured, and which contains the processing means. In FIG. 1, the user interface for showing feedback is in a computer 33. Alternatively, a smart phone 33.1 can be used, in which the accuracy of the display is at least 640×360 pixels, in order to clearly display a multi-line feedback series.

A combination of a PPG wrist-worn device, equipped with an acceleration sensor, and a smart phone has proven to be technically the best choice. With a large display, a smart phone can show several feedbacks simultaneously. It has powerful processing means and utilizes the data of the positioning device and the said acceleration sensor. Using an acceleration sensor, the reliability of measurement using a PPG sensor (photoplethysmogram) improves sufficiently, but the acceleration-sensor's data can also be used to identify a context, as can positioning data.

The processing means 38 of the apparatus of the system according to the invention are arranged to analyse the recorded measurement data in order to define the values of the preselected variables, to define at least one temporal context 24 from the analysed measurement data, and to select the feedback series 12, 12′, 12″ to be used, according to the defined contexts 24. In addition, the processing means are arranged to classify the value of each preselected variable in a value range corresponding to it, to pick feedback 18 corresponding to this value range from each feedback series 12, 12′, 12″, to arrange the picked feedbacks 18 in an order according to the priority rules thus forming a group of consecutive feedbacks 18, and to show a number of picked consecutive feedbacks 18, according to the limit rules, to the person 20, with the aid of the user interface 23.

In its simplest form, the entire apparatus can be situated in the monitor 34 performing the measurement, through the user interface of which feedback is also given to the user. The user interface 23 for entering the initial parameters 25 can also be in the measuring device, but the initial parameters are preferably entered with the aid of the user interface in the computer. In this connection, the term initial parameters refers to the preselected feedback series of the method, the feedbacks they contain, and the variables and sets of rules to be used. Between the monitoring device and the computer comprising the apparatus data transfer can take place wirelessly, for example with the aid of Bluetooth or WLAN (WiFi), or in a wired form. Alternatively, the monitor can be only for physiological measurement, in which case the apparatus comprising the processing means can be situated in the computer 33 or in the internet. The computer 33 can be connected to the internet. A further alternative is for the apparatus to be located in the internet, when the processing means will be located on a server and the user interface 23 will be on an internet 44 website and can be used through a web browser, for example, from a mobile phone, a tablet, or a computer.

Next, the operation of the method according to the invention will be described in an example according to one embodiment. FIG. 2 shows the method according to the invention in a simplified form. In the method, physiological measurement is performed in step 212 and a second measurement in step 213. The second measurement comprises the measurement of some variable depicting the external state of the person, such as acceleration or position. A physiological analysis of the data recorded from these measurements is performed in step 214. The variable of the external state is also used to ascertain the reliability of the physiological measurement. A high heart-rate value is probably an error if the acceleration is zero. In addition, temporal contexts are defined from the measurement data in step 216. The values of the selected variables obtained as a result of the analysis are placed in unique value ranges of feedback series classified as contexts, in step 220, giving as a result feedbacks using preselected rules. These feedbacks are prioritized in step 224 and the most important are shown to the person according to the limit values in step 226. The performance of the method requires the creation of preselected feedback series and priority rules for the feedbacks in steps 202 and 204, before the measurement data can be analysed. The feedback series and feedback priority rules can be brought to the method according to FIG. 2 when the values of the variables are placed in the feedback series, or, according to FIG. 3, as a separate step 210 before performing the measurement.

According to FIG. 3, an expert system is used in the method, in which the necessary sets of rules and feedback series containing feedback are defined before using the method. Each feedback series comprises a group of mutually exclusive feedbacks, according to step 204. There can be a large number of feedback series, for example, one hundred of them. The feedback series are question groups relating to one or more variable or variables monitored from physiological measurement data and divided into subject areas. The feedbacks and feedback series are formed based on general expert knowledge of the interpretation of a physiological state.

An example of a feedback series can be the feedback series 12, 12′, 12″, relating to the numerical value of training effect (i.e. TE) depicting the effectiveness of exercise training, which is shown in FIG. 8. In the feedback series 12 there is a group 14 of questions, for example “is TE≧1”, “is TE≧2”, “is TE≧3”, and “is TE≧4”. In this case, the value ranges of the variable TE are thus “0≦TE<1”, “1≦TE<2”, “2≦TE<3”, “3≦TE<4”, and “TE≧4”. Each question can be answered “yes” or “no”, and the answer gives one feedback or alternatively the examination moves forwards to the next feedback series. Each question has preferably its own feedback. The feedbacks are mutually exclusive in that, for example, the sleep time measured from a measurement period cannot be simultaneously good and poor. The mutual exclusiveness of the feedbacks refers to the fact that there cannot be two feedbacks from the same feedback series in the feedback to be shown to the person. A feedback series can sometimes also consist of only a single feedback. Two separate feedbacks can then be given from questions concerning the same variable, for example, “is TE≧3” and “is TE≧4”, as the questions are then in different feedback series. As one feedback series contains, for example, from one to ten feedbacks, the total number of feedbacks can be several hundreds, even thousands, so that it can be easily understood that the number of feedbacks to be shown must be limited.

The rules to be set for the database can be, for example, rules as to which of the feedback series belong to the context “sleep”. The feedback series of the context “sleep” can be, among others, the duration of sleep, respiratory frequency, the time needed to go to sleep, and movement during sleep. Another rule can define context-specifically the order of importance of the feedback series in the context “sleep”, which could be, for example, 1) duration of sleep 2) time needed to go to sleep 3) movement during sleep, and 4) respiratory frequency. Preferably at least some of the rules are context-dependent.

The feedback 18 to be picked from each feedback series depends on the value of the variable being examined in the feedback series, obtained from the value range. Thus, the feedbacks contained by an individual feedback series generally mutually differ from each other. In the method, in step 202 the rules of the expert system are used to prioritize and show to the person the feedbacks 18 given by the feedback series 12, 12′, 12″. The rules can contain, for example, the feedback series to be used context-specifically and the order of prioritization of the picked feedbacks, as has been described earlier. Variables to be monitored from the measurement data are also selected for the database; these can be, for example, oxygen consumption, stress, respiration, recovery, TE, and other corresponding variables that assist the physiological analysis.

The physiological measurement itself can be performed in step 212 shown in FIG. 4, in which the person's state is monitored continuously, recording the measurement of the heart rate to form the measurement data. In the physiological measurement, heart rate and, at the same time, a second variable such as acceleration, are measured. The measurement of heart rate takes place with the aid of, for example, a heart-rate band, ECG electrodes, or a PPG meter, directly or indirectly. Direct measurement refers to measurement, which gives directly a heart-rate value, whereas indirect refers to measurement, which gives indirectly heart rate, for example, from the training's intensity data. The person's movement and/or positioning data is preferably measured as the second variable. Movement can be measured with the aid of an acceleration sensor and positioning data, in turn, with the aid of, for example, a GPS device or mobile phone.

Physiological measurement is preferably performed continuously or partly periodically, by monitoring the person's normal life, i.e. the person carries the heart-rate-measuring device with them in everyday life. In this case, the term everyday life refers to monitoring that takes place outside controlled laboratory conditions. The term partly periodical measurement refers to the fact that there can be short breaks in the measurement, but the depiction of the physiological state of the person obtained through the measurement is continuous and reliable. The physiological data obtained in this way depicts the person's normal life comprehensively, unlike measurements performed in laboratory or controlled conditions, which are known from the prior art. The measurement data obtained from the physiological measurement can be recorded, for example, in the memory of the monitor measuring heart rate, or it can transmitted over a network to a computer or be stored in a cloud service.

After performing physiological and state-data measurement, a physiological analysis is performed on the recorded measurement data in step 214 of FIG. 4, which includes, among other things, the identification of stress, recovery, and exercise periods from the heart-rate data and the formation of the values of their variables. FIG. 5 shows one possible heart-rate curve 300. Stress is a natural reaction by the body, with the aid of which the body seeks to respond to the demands of the environment. The activity of the autonomic nervous system is then dominant while that of the parasympathetic nervous system is recessive. This appears as, for instance, a rise in heart-rate level and in respiratory frequency. The factor causing a stress reaction can be mental, physical, or social, and can have a positive or negative character. Recovery refers to the relaxation of the body and/or a reduction in the level of activity, for example, during relaxation, rest, and calm work. The parasympathetic activity of the autonomic nervous system is then dominant, i.e. the heart-rate level is low and respiration is relaxed.

Stress affects the psychophysiological regulation of the body, for instance, through the autonomic nervous system. In stress measurement, when evaluating the stress state, it is essential to differentiate the facts affecting autonomic regulation and exclude, for example, rises in the activity level caused by exercise. The present method is able to differentiate different physiological states by combining heart-rate, oxygen-consumption, and respiratory frequency data with each other. This reveals information as to when the body's activity level is raised due to the effect of exercise and when due to the effect of mentally-loading factors. When evaluating the activity of the autonomic nervous system, the connection of heart-rate to other physiological factors can also be taken into account. Heart-rate is affected by, among other things, metabolic processes, posture and changes in posture, respiratory rhythm, physical activity, emotions and thoughts, and stress and recovery.

The physiological analysis contains several different steps: processing of the heart-rate signal, formation of physiological variables, portioning of heart-rate data, exclusion of physical activity, identification of stress states and recovery states, and formation of the values of variables. In the physiological analysis, several computational steps are performed in a specific order. The steps can be defined as follows: (1) initial variations in the ECG and/or heart-rate signals; (2) segmentation of the heart-rate signal into stationary segments; (3) identification of segments raising the heart rate, which relate to something other than stress, including physical training, physical activity, recovery from physical activity, and changes in posture; (4) identification of segments relating to a relaxed state; (5) identification of segments containing a potential stress state; and (6) combination of the information collected in steps 3-5 in order to create an overall index depicting stress. The physiological analysis can also comprise the setting of certain initial parameters, such as minimum heart rate, whereas some of the properties can be identified directly from the measured data, or entered manually. If required, manually-set background data on the person being measured, such as age, height, weight, and sex, can also be entered in the physiological analysis. However, these are not essential in all situations.

In other words, in the physiological analysis, the heart-rate signal is segmented into physiological states, in which in the method the heart-rate signal is segmented to form internally coherent segments and in which in the method at least one analysis is used to identify segments with an increased metabolic rate due to training, for example, physical activity, movement, or change of posture. The analysis can be performed, for example, by collecting data on i) repeated heart-rate changes and HRV measurements, such as moving co-variances appearing in the selected embodiments, ii) HRV measurements, or the components in them, for example, the LF and HF components, iii) training intensity, such as changes in the heart rate and/or the effect of respiratory periods on oxygen consumption, iv) recovery from training, v) respiratory periods or ventilation together with the heart rate and/or HRV, or the divergence of the heart-rate level, vi) use of the information on the temporary properties of training, physical activity, or movement, specific to a frequency or time-definition group, or vii) the use and combination of information obtained from several definitions. The physiological analysis and its steps are described in greater detail in the applicant's patent EP 1545309.

In the first step of the physiological analysis, various variables are formed to depict the activity of the autonomic nervous system and the body's physical activity level. Disturbances in the heart-rate interval signal due to measurement are corrected and it is pre-processed using various digital filters, in order to improve the quality and interpretation of the signal. Respiratory variables (respiratory frequency, ventilation, and oxygen consumption) among other things are calculated from the heart rate. In addition, various heart-rate-variation variables and autonomic-nervous-system parameters are defined from the heart-rate data, on the basis of which the activity states of the sympathetic and parasympathetic nervous systems can be defined.

In the second stage of calculation, the data calculated in the first step are combined, in order to define stress reactions and relaxation states. Oxygen consumption is a measure of the body's physical activity, which can be utilized to exclude, from the detection of a stress state, moments in time in which the state of the autonomic nervous system is excited due to physical stress or recovery after training. Once the analysis has excluded excitation of the autonomous nervous system due to physical activity, loading due to other reasons is evaluated.

The stress level and relaxing moments in time are defined for the remaining moments in time, utilizing the model of the activity level of the autonomic nervous system, formed on the basis of the heart-rate variation and heart-rate level, and the respiratory variable. The stress level can be scaled separately for each person, so that improved discrimination will be achieved in the monitoring of an individual. The stress level can be defined separately for each moment in time. As a result of the physiological analysis, the physiological states of the person are determined for the measurement period, as well as values for the selected variables depicting the intensity and direction of the variable in question. FIG. 6 shows a heart-rate curve divided into physiological states 50, of which 52 is stress, 54 is recovery, and 56 is exercise. A complete description of the detection of stress, recovery, and physical activity is to be found in the applicant's U.S. Pat. No. 7,330,752 B2.

Once a physiological analysis has been performed on the measurement data, we can move to step 216 of FIG. 4, in which the temporal contexts are defined from the measurement data. A context can be defined either manually as an optional input 218 with the aid of diary entries, or automatically detected from the data. It is also possible, in the case of the same data, for some of the contexts to be identified automatically and some to be set manually. If the context is defined manually, information is entered in the calculation as to when, for example, is sleep time, leisure, exercise, and worktime. Information can be entered through the user interface of the device or apparatus. The temporal contexts can also overlap, such as exercise often overlapping with leisure or worktime. However, it is preferable to perform the definition of the contexts automatically by analysing the measurement data, in which case external input will not necessarily be required at all.

According to one embodiment, the definition of contexts can use automatic optional input, which can be, for example, the context information “on phone”, “listening to music”, “browsing internet”, “calling friend”, or “travelling in bus”, which is automatically available from a mobile application. The definition of context can then also be performed in some cases entirely without the contexts being defined on the basis of the measurement data.

The automatic identification of temporal contexts is described next. The exercise context is defined automatically by utilizing heart-rate and/or velocity data. On the basis of heart-rate variation, oxygen consumption is modelled, which depicts the intensity of loading. If oxygen consumption is more than 30% of the person's maximal performance, the context is identified as exercise. To identify exercise, motion data can also be utilised, with the aid of which different exercise forms, such as walking, running, and cycling, can also be identified. The automatic interpretation of exercise is based on the methods disclosed in the applicant's publications U.S. Pat. No. 7,330,752 B2 and US2006032315 A1.

Sleep time can be defined on the basis of acceleration and/or heart-rate data. The person's posture and amount and direction of movement are identified from the acceleration signal. If the person is prone, and does not move for a sufficient length of time (little movement), it can be decided that the person is asleep. Correspondingly, sleep time ends when movement appears for a sufficient length of time. Identification can be improved by combining motion and heart-rate data. At the moment of going to sleep, the heart rate decreases and the heart-rate-interval variation increases considerably. The precise time of going to sleep can be identified on this basis. Worktime and other position-specific contexts can be defined automatically on the basis of GPS data. For example, worktime and when the person is at home can be picked out from the GPS data. Worktime is based on, among other things, the assumption that when the person is in the vicinity of their workplace, they are at work. More corresponding position-specific context definitions can be made, for example, in such a way that the person sets information for the GPS device concerning the position that they are in the vicinity of when at home, shopping, at the gym, or in other similar places. Leisure can be identified after identifying worktime and sleep time. If worktime is not identified, leisure is regarded as being all other time apart from sleep time. FIG. 7 shows an example, in which the daily measurement is divided into contexts 24.1-24.4.

After the definition of the contexts, the value of each variable defined from the measurement data is classified in the value range of a feedback series defined in the database of the processing means, according to step 220 of FIG. 4. The feedback series are preferably classified context-specifically, so that in each context only those feedback series that are sensible in terms of the examination of the relevant context are used. For example, in the temporal context “worktime” it is not sensible to use feedback series that include questions dealing with sleep. In this connection, the term classification of the values of the variables refers to a value range 22, which is located in the feedback series in order to pick a feedback according to step 222, being formed from the sliding value of the value of the variable. For example, the value of the variable training effect TE can be, with reference to FIG. 8, 2,7, which is classified in the third value range 22 (number 3). In turn, this value range 3 gives the answer “yes” to one question in the feedback series' group, when one selected feedback is obtained from the feedback series in question. More specifically, in this embodiment, the value of the variable is situated in consecutive questions in the preselected priority order in the feedback series and in the case of an answer “yes” we move to the next feedback series.

Alternatively, the value of the variable shown in FIG. 8 could be situated in a question in each feedback series, if the question were to be set in such a way that only one question from each feedback series could receive the answer yes. For example, in the case of FIG. 8, the value range 1 would contain the condition TE≧4, the value range 2, in turn, the condition 4>TE≧3, the value range 3 the condition 3>TE≧2, and finally the value range 1, in turn, the condition 2>TE≧1.

In this connection, the term value range refers to the address of the memory location of a specific feedback, from which a selected feedback is picked using the value of a variable. The feedback series being processed can relate, for example, to sleep quality, the duration of sleep, the timing and amount of recovery, the timing and amount of stress, the timing and amount of exercise, the intensity of exercise, and possibly also to the use of alcohol. Thanks to the great calculating power of computers, all the feedback series can be processed in each context, but this is not sensible, as some of the feedback series relate essentially to a specific temporal context.

Because many different feedback series can often be defined from the data, and it is not reasonable to provide feedback from all the feedback series, the feedback should be prioritized. Prioritization can take place in three different steps. In the first step, prioritization takes place in each context. For example, in the context “exercise” the feedbacks relating to exercise are primary, feedbacks relating to leisure are secondary, and last of all come feedbacks relating to worktime.

In the second step, the realized conditions given by the feedback series of the various contexts are combined according to a preselected feedback direction. For example, if it is wished to provide feedback with an emphasis on exercise, the feedbacks relating to variables depicting exercise then receive a higher order of importance than other feedbacks. A feedback direction can be either set manually by the user, or it can be defined in the set of rules according to the device providing the feedback. There can be many different alternative feedback directions, but the exercise-weighted subject-area emphasis of FIG. 8 and the stress-management-weighted subject-area emphasis of FIG. 9 can act as examples. In exercise-weighted prioritization, the feedback series 12, 12′, 12″ relating to exercise and other physical activity, and their feedbacks 18, are given, according to the preset feedback direction, priority over feedbacks 18 relating to stress and recovery. In the stress-management-weighted feedback direction, the situation is the reverse. By means of this prioritization, the aim is to provide the user with the most interesting and topical feedback. Though there can be many feedback directions, the intention is for there to be considerably fewer feedback directions than there are feedback series in general. The desired feedbacks can then be picked, according to the priority rules of the feedback direction, from the feedbacks given by the feedback series situated in contexts, arranging from the feedbacks prioritized groups of consecutive feedbacks according to step 224 of FIG. 4. These consecutive feedbacks are shown to the person, according to the preselected limit rules, in step 226.

In the exemplary subject areas of FIGS. 8 and 9, the feedback series 12, 12′, 12″ are grouped according to prioritization, so that the feedbacks 16 obtained from them are directly in the selected consecutive order. In them, the conditional-statement series are directly in the desired order, i.e. the prioritization order is included in this. Once the first feedback series 12 has been gone through, we move to the next feedback series 12′ in the priority order, and after that to the third feedback series 12″. An individual question of the feedback series can include a question relating to one or more variables, for example, “is TE<2 and is the duration of exercise<30 min?”, when the question is of a vector. Here too, the value ranges of the combinations of variables are mutually exclusive. Only one combination is realized at any one time. In addition, it can be defined in the preselected rules that in a certain value range a specific feedback series ends and a change is made directly to the next feedback series, or that some feedback series is omitted.

Next, the example of FIG. 9 is described in detail from a mathematical point of view. Other variables are connected to the actual physiological variable, often from the said other source, which permits the use of the value of the variable only when conditions are appropriate for its use. If we examine only the first vertical feedback series 12 in the figure, we note that the variable relating to the feedback series is a vector, which comprises three scalar variables A) “Measurement contains a period of sleep?” B) “Measurement error” and C) “Recovery”. In this case, the scalar variable A) is discrete, as the measurement either contains a period of sleep or does not, i.e. the possible value ranges are 0 or 1. For its part, the scalar variable B) is continuous in two different value ranges, i.e. 0-<15% and 15-100%. The scalar variable C) is continuous in four different value ranges, i.e. 0-<25%, 25-<50%, 50-<75%, and 75-100%. The vector formed by the scalar variables can thus obtain the following value ranges, which are formed of the value ranges obtained by the scalar variables, as well as the mutually exclusive feedbacks corresponding to the value ranges:

[0,-,-]->no feedback, next feedback series

[1,0-<15,-]—feedback #10a

[1,15-100,0-<25]—feedback #10e

[1,15-100,25-<50]—feedback #10d

[1,15-100,50-<75]—feedback #10c

10 [1,15-100,75-100]—feedback #10b

In this connection, the marking “-” in the value range of the scalar variable refers to the fact that this scalar variable is not processed, as in this case processing is not possible or appropriate on the basis of a previous answer. If, for example, in the case of FIG. 9 the scalar variable A) receives the value 0, i.e. the period of sleep does not form part of the measurement, it is also not possible to determine to what extent the period of sleep has contained measurement error or recovery.

Preferably the method according to the invention further includes the selection of the feedback direction before the feedbacks are shown to the person, when feedbacks are picked according to the selected feedback direction to form prioritized groups. Thanks to this, when the person selects exercise-weighted feedback as the feedback direction, they can be shown, for example, three feedbacks from an exercise-weighted feedback direction and one feedback from a stress-weighted feedback direction.

According to one embodiment, the selection of the feedbacks to be shown also includes a third step, in which the number of feedbacks is limited according to the type of feedback-provision device used to show feedback to the person. This limitation of the number of feedbacks is preferably device-specific. In FIG. 10 it is determined, for example, whether the feedback-provision device is a heart-rate meter or a mobile device. If the feedback-provision device is a heart-rate meter, only a single feedback statement can be given, due to the small display. The content of the feedback statement in question also depends on the realized situation, and on the weighting according to which the feedback is given. If the emphasis is exercise-weighted, the user is given a specific feedback statement #1b according to FIG. 10, which provides feedback on their exercise performance. Though, on the basis of the exercise-weighted prioritization, the prioritized group would also have included two other feedbacks #2c and #3a, feedback #1b shown according to the exercise-weighted internal priority order takes priority over them. However, if the question is of a mobile device, in which, according to the initial setting, it is possible to give a maximum of 2 exercise weighted and 1 stress-management-weighted feedbacks, feedbacks #1b, #2c, and #10d are given. If, in turn, stress-management-weighted prioritization has originally been selected, feedback is provided correspondingly, but, according to the weighting, stress and recovery related feedbacks take priority over exercise feedbacks.

In the prioritization of feedbacks a statistical method can also be used, in which the questions of each feedback group are given a statistical probability in the range of, for example, 0-100 and the feedbacks to be shown are prioritized on the basis of the probability. The final result corresponds to the use of prioritization and limit rules.

TABLE 1
Example of implementation.

| | | | | | | Selected | | | Feedbacks |
|---|---|---|---|---|---|---|---|---|---|
| | | Feedback | | Feedback | | feedback | | | to be |
| Variables | Contexts | series | Feedbacks | directions | Feedback | direction | Feedback | Device | shown |
| Sleep time | Sleep | | | | | | | | |
| Oxygen | | 1. | A | Exercise | | Exercise | N | PC | n, o, p, e |
| consumption | | | | | | | | | |
| | | 2. | B | | N | | O | Heart- | n |
| | | | | | | | | rate | |
| | | | | | | | | meas. | |
| Stress | | 3. | C | | O | | P | | |
| TE | | 4. | D | | P | | E | | |
| Respiration | Work | | | | K | | | | |
| | time | | | | | | | | |
| Recovery | | 5. | E | Stress | | Stress | E | PC | e, f, n, b |
| | | 2. | F | | E | | F | Heart- | e |
| | | | | | | | | rate | |
| | | | | | | | | meas. | |
| | | 6. | G | | F | | N | | |
| | | 7. | H | | L | | B | | |
| | | 8. | I | | D | | | | |
| | Leisure | | | | G | | | | |
| | | 9. | J | | | | B | | |
| | | 10. | K | | | | C | PC | b, c, e, f |
| | | 6. | L | Recovery | | Recovery | E | Heart- | b |
| | | | | | | | | rate | |
| | | | | | | | | meas. | |
| | | 7. | M | | B | | F | | |
| | Exercise | | | | C | | N | | |
| | | 11. | N | | I | | | | |
| | | 9. | O | | M | | | | |
| | | 10. | P | | Q | | | | |
| | | 12. | Q | | | | | | |
| | | 13. | R | | | | | | |

Table 1 shows an example of the selection and prioritization of feedback in different contexts. The table shows the variables to be monitored from the physiological data only as a listing, with no context or feedback series allocated to them. The contexts “sleep”, “work time”, “leisure”, and “exercise” are differentiated using the measured measurement data. According to predefined conditions, the contexts include feedback series 1-13. In this connection, it should be understood that the same feedback series can appear simultaneously in several contexts and the feedback given the specific value of a variable in each context of the feedback series in question can vary context-specifically. For example, the value “30” of the variable respiration can give the feedback “you are lively” in the context “work time”, but in the context “exercise” the feedback “training is too light”. The value of the variable gives a certain value range, on the basis of which the feedbacks a-r are picked. The picked feedbacks can be formed into prioritized groups according to subject area and selected feedback direction. According to the table of FIG. 10, for example, the feedbacks n, o, and p can be selected from the context “exercise” and feedback k from the context “leisure” from the priority weighting “exercise”. Further the selected feedback direction “exercise” can select three feedbacks (n, o, and p) from the previous four and take one feedback (e) from the priority weighting “stress”. The prioritized group would then be the feedbacks n, o, p, and e. After this, the feedback to be shown to the person can be prioritized further according to the feedback-providing device, so that, for example, using a computer, all four feedbacks (n,o,p,e) are shown from the feedback to be shown, whereas, using a heart-rate meter, only the feedback n that has the highest priority is shown.

Feedback can be given in many different ways, for instance, with the aid of a device, a mobile application, or a PC program, and in many different time windows, either in real time, or through later analysis. Feedback can be given, for instance, in connection with a daily measurement graph by referring to specific moments in time, according to FIG. 11, or as a summary in connection with the graph of averaged stress and recovery over a specific period of time. In this case, all the feedbacks given during a day will fit simultaneously on the display of a smart phone. Alternatively, feedback can be shown at the end of a specific context, such as exercise or sleep time, by comparing a specific moment, day, week, or month to earlier situations and giving comparative or summing feedback, or by summing the events of a desired moment in time, and reporting their effects, for example, on health. Feedback can be given graphically or also as a sound recording. In all cases, the feedback is very clear and concise.

The use of the feedback series can depend on the amount of recorded data, which can be applied in both the priority and limit rules. It is easy to understand that a small amount of data will reduce the reliability particularly of some physiological variables, in which case they should not be used. A small amount of data can also be a reason to limit the number of feedbacks.

1-16. (canceled)

17. A method for providing computer-aided feedback to a person with the aid of a monitor and an expert system, comprising:
feedback series to be used with the aid of software and relating to a temporal context, and a set of rules to be implemented with the aid of software for selecting feedbacks from the feedback series, and
in which method each feedback series comprises a group of mutually exclusive preselected feedbacks, each feedback relating to a unique value range of a preselected variable, and
in which method the providing of feedback includes the following steps:
monitoring a state of the person by a monitor for at least a portion of a day, with the monitoring being recorded as measurement data and the said monitoring including the measurement of heart rate, using a first sensor, and the measurement of another input,
analysing the recorded measurement data, in order to determine the values of the preselected variables,
determining at least one temporal context from the analysed measurement data,
selecting the feedback series to be used according to each selected temporal context,
classifying the value of each preselected variable into one said value range and picking a feedback corresponding to this value range from each feedback series, wherein in which the set of rules includes priority rules for prioritizing the selected feedback consecutively, and limit rules for selecting the number of feedbacks to be shown at one time to the person from the consecutive feedbacks,
in which method the providing of feedback further includes the following steps:
arranging the picked feedbacks in an order according to the priority rules, thus forming a group of consecutive feedbacks, and
picking the consecutive feedbacks from the group of consecutive feedbacks, the number of consecutive feedbacks displayed being limited determined to the limit rules,
displaying on a chosen device the consecutive feedbacks for viewing by the person.

18. The method according to claim 17, wherein the variable relating to the feedback series (12, 12′, 12″) is a vector comprising two or more scalar variables.

19. The method according to claim 17, wherein the limit rules include logic depending on the chosen feedback-provision device.

20. The method according to claim 19, wherein, the feedback directions, to be selected by the user, are formed, each feedback direction including preselected feedback series selected according to the feedback direction.

21. The method according to claim 20, wherein the feedback alternatives selected in each feedback direction are set feedback-direction-specifically in a priority order according to the preselected rules.

22. The method according to claim 17, wherein the measurement data is analysed, in order to find physiological states, in the following steps:
the measurement data is corrected to eliminate disturbances and is pre-processed,
the values of the variables are formed on the basis of the corrected and pre-processed measurement data,
stationary states are segmented from the measurement data
states of physical activity are detected using first preselected criteria,
the measurement data is compared to other known physiological states, and
the physiological states are identified using second preselected criteria.

23. The method according to claim 17, wherein the other input includes at least one of the following: positioning of the person, acceleration measurement, temperature of the person, ambient temperature.

24. The method according to claim 17, wherein the use of at least one feedback series depends on the amount of recorded data.

25. The method according to claim 24, wherein the feedback series are implemented as conditional-statement groups and they are set to be realized consecutively, so that the said consecutive feedbacks are obtained in this order.

26. The method according to claim 17, wherein heart rate is measured using a PPG wrist-worn device equipped with an acceleration sensor, the acceleration data of which is also used to differentiate the contexts.

27. The method according to claim 17, wherein the said apparatus and user interface are detected automatically for determining the number of feedbacks.

28. A computer-aided system for providing feedback to a person on the basis of monitoring, comprising:
a monitor for continuously monitoring the person's heart rate and state and recording the measurement data obtained, and an apparatus having:
processing means for processing, by means of software, feedback series relating to a temporal context, in order to select feedbacks according to a preset set of rules, and
a user interface for input by the user and for displaying selected feedbacks to the person,
in which each feedback series comprises a group of mutually exclusive feedbacks, each feedback relating to the value range of a preselected variable,
in which the processing means are arranged to perform the following steps:
to analyse the recorded measurement data in order to determine the values of the preselected variables,
to define at least one temporal contexts from the analysed measurement data,
to select the feedback series to be used, according to the defined contexts,
to classify the value of each preselected variable to a value range corresponding to it, wherein the set of rules includes priority rules for prioritizing the selected feedbacks consecutively, and limit rules for selecting, from the consecutive feedbacks the number of feedbacks to be displayed to the person at one times, and in which the processing means are arranged to perform the following steps:
to pick a feedback corresponding to this value range from each feedback series,
to arrange the picked feedbacks in an order according to the priority rules, thus forming a group of consecutive feedbacks,
to display a number of picker consecutive feedbacks, according to the limit rules, to the person with the aid of the user interface.

29. The system according to claim 28, wherein the processing means include software means for the selection of the feedback direction for the person and for selecting the preselected feedback series according to the selected feedback direction.

30. The system according to claim 28, wherein the monitoring apparatus for monitoring the person includes an acceleration sensor and/or position-detection means.

31. The system according to claim 28, wherein the monitor comprises tape-attached ECG sensors and an electronics unit supported by them.

32. The system according to claim 28, wherein the system comprises a PPG wrist-worn device equipped with an acceleration sensor and a large-display smart phone connected to each other over a wireless link, and in which there is also a positioning device (GPS).
